(19) United States
(12) Patent Application Publication
(10) Pub. No.: US 2006/0147983 A1
(43) Pub. Date: Jul. 6, 2006

(54) Nucleic acid detecting sensor, nucleic acid detecting chip, and nucleic acid detecting circuit

(75) Inventor: Shin-ichi O'uchi, Tsukuba-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOSHIBA, Minato-ku (JP)

(21) Appl. No.: 11/366,472

(22) Filed: Mar. 3, 2006

Related U.S. Application Data
(63) Continuation of application No. PCT/JP05/19358, filed on Oct. 14, 2005.

(30) Foreign Application Priority Data
Oct. 14, 2004 (JP) 2004-300267

(51) Int. Cl. C12Q 1/68; C12M 1/34

(52) U.S. Cl. 435/6; 435/287.2; 977/702

(57) ABSTRACT

Nucleic acid detecting sensor includes field-effect transistor, detector which detects target nucleic acid molecules having sequences from sample based on degree of a variation in threshold voltage of field-effect transistor, and at least one nucleic acid probe molecule which is hybridized with corresponding one of target nucleic acid molecules, and is immobilized on gate of field-effect transistor, wherein gate width of field-effect transistor is of order of length obtained by expression given below (ε₀εᵣk_{B}T/e²n)^{1/2} where ε0 is dielectric constant of vacuum, εr is relative dielectric constant of channel region, kB is Boltzmann constant, T is absolute temperature of the channel region, e is elementary charge, and n is equilibrium carrier density in the channel region in field-effect transistor where channel is formed.

### CROSS-REFERENCE TO RELATED APPLICATIONS

This is a Continuation Application of PCT Application No. PCT/JP2005/019358, filed Oct. 14, 2005, which was published under PCT Article 21(2) in English.

This application is based upon and claims the benefit of priority from prior Japanese Patent Application No. 2004-300267, filed Oct. 14, 2004, the entire contents of which are incorporated herein by reference.

### BACKGROUND OF THE INVENTION

1. Field of the Invention

The present invention relates to a nucleic acid detecting sensor that detects a target nucleic acid molecule included in a sample using a field-effect transistor (FET), a nucleic acid detecting chip, and a nucleic acid detecting circuit.

2. Description of the Related Art

Conventionally, there has existed a nucleic acid detecting sensor that detects whether a target nucleic acid molecule is included in a sample using an FET (see, for example, Toshiya Sakata et al., “Detection of DNA Hybridization using Genetic Field Effect Transistor,” Extended Abstract (The 64^{th} Autumn Meeting 2003), p. 1179; Jpn. Pat. Appln. KOKAI Publication No. 2003-322633; PCT National Publication No. 2001-511246, etc.).

Conventionally, however, there is no method of detecting a signal of one nucleic acid molecule with efficiency or no technique of conducting a quantitative analysis within a wide density range, using an FET.

### BRIEF SUMMARY OF THE INVENTION

In accordance with a first aspect of the invention, there is provided a nucleic acid detecting sensor comprising: a field-effect transistor; a detector which detects target nucleic acid molecules having sequences from a sample based on a degree of a variation in threshold voltage of the field-effect transistor, and at least one nucleic acid probe molecule which is hybridized with a corresponding one of the target nucleic acid molecules, and is immobilized on a gate of the field-effect transistor,

wherein a gate width of the field-effect transistor is of an order of a length obtained by an expression given below:
(ε₀εᵣk_{B}T/e²n)^{1/2}
where ε₀ is a dielectric constant of a vacuum, εᵣ is a relative dielectric constant of a channel region, k_{B} is a Boltzmann constant, T is an absolute temperature of the channel region, e is elementary charge, and n is an equilibrium carrier density in the channel region in the field-effect transistor where a channel is formed.

In accordance with a second aspect of the invention, there is provided a nucleic acid detecting sensor comprising: a field-effect transistor; a detector which detects target nucleic acid molecules having sequences from a sample based on a degree of a variation in threshold voltage of the field-effect transistor; and at least one nucleic acid probe molecule which is hybridized with a corresponding one of the target nucleic acid molecules, and is immobilized on a gate of the field-effect transistor,

wherein a gate length of the field-effect transistor is of an order of a length obtained by an expression given below:
(ε₀εᵣk_{B}T/e²n)^{1/2}
where ε₀ is a dielectric constant of a vacuum, εᵣ is a relative dielectric constant of a channel region, k_{B} is a Boltzmann constant, T is an absolute temperature of the channel region, e is elementary charge, and n is an equilibrium carrier density in the channel region in the field-effect transistor where a channel is formed.

### BRIEF DESCRIPTION OF THE SEVERAL VIEWS OF THE DRAWING

FIG. 1 is a perspective view showing one of nucleic acid detecting sensors according to an embodiment of the present invention, which arranged on a nucleic acid detecting chip;

FIG. 2 is a diagram showing an example of a nucleic acid detecting circuit that detects a nucleic acid using the nucleic acid detecting sensor shown in FIG. 1;

FIG. 3 is a flowchart showing an operation of the nucleic acid detecting circuit shown in FIG. 2;

FIG. 4 is a circuit diagram showing a nucleic acid detecting circuit as a modification to the circuit shown in FIG. 2;

FIG. 5 is a chart showing the principle of quantitative analysis;

FIG. 6 is an illustration of a configuration of sensors on a chip to conduct quantitative analyses for multiple kinds of nucleic acids;

FIG. 7 is a diagram of a nucleic acid detecting circuit using a differential amplifier as another modification to the circuit shown in FIG. 2;

FIG. 8 is a diagram of a nucleic acid detecting circuit using a differential amplifier as still another modification to the circuit shown in FIG. 2; and

FIG. 9 is a diagram of a nucleic acid detecting circuit using a double gate MOSFET as yet another modification to the circuit shown in FIG. 2.

### DETAILED DESCRIPTION OF THE INVENTION

A nucleic acid detecting sensor, a nucleic acid detecting chip and a nucleic acid detecting circuit according to an embodiment of the present invention will be described in detail with reference to the accompanying drawings.

The embodiments of the present invention have been developed in consideration of the above situation and its object is to provide a nucleic acid detecting sensor using an FET, a nucleic acid detecting chip and a nucleic acid detecting circuit, which are drastically improved in sensitivity.

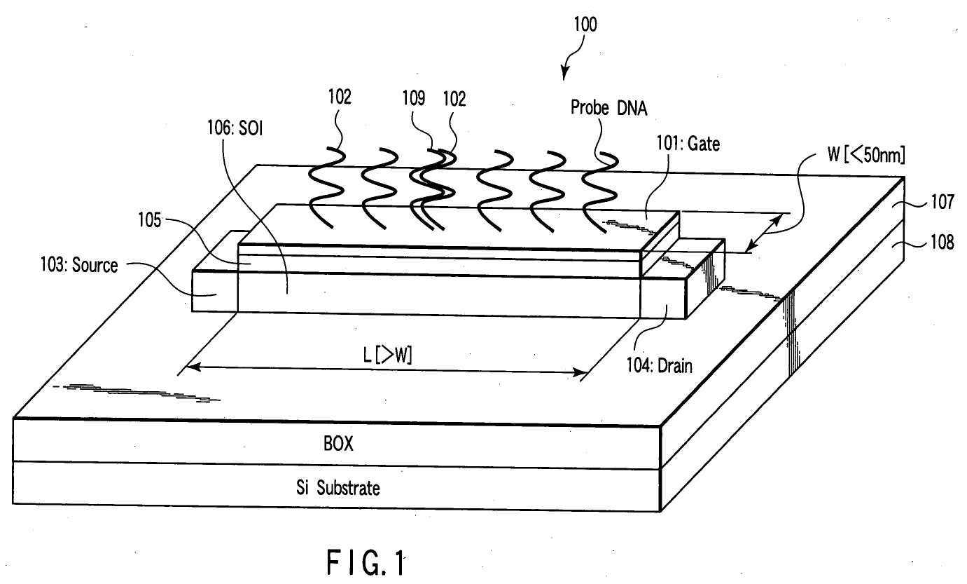

The nucleic acid detecting circuit according the embodiment of the present invention comprises a nucleic acid detecting sensor 100. The sensor 100 includes a metal oxide semiconductor field-effect transistor (MOSFET) and a substrate. Usually, a plurality of nucleic acid probe molecules (probe DNA) 102 are immobilized to the MOSFET. The MOSFET has a gate 101, a source 103 and a drain 104. The nucleic-acid-probe molecules 102 are immobilized onto the gate 101. As shown in FIG. 1, the source 103 and drain 104 are connected to each other via a body 106, and the gate 101 is stacked on the body 106 with a gate oxide film 105 interposed therebetween. The source 103, drain 104 and body 106 are provided on a buried oxide (BOX) 107. The sensor 100 can be manufactured using a wafer having a silicon on insulator (SOI) structure as shown in FIG. 1, and the same can be done using a bulk silicon (Si) substrate, which will be understood by one of ordinary skill in the art.

The nucleic acid detecting circuit according the embodiment of the present invention determines whether a target nucleic acid molecule is detected based on the degree of modulation of electrical properties of the MOSFET. In the embodiment, the gate 101 is elongated in which direction the source 103 and drain 104 are connected and, in other words, the gate 101 is decreased in gate width W. Since the electrical properties of the MOSFET is greatly modulated even by the variation of a small number of charges caused on the gate 101, the circuit can also detect a small number of target nucleic acid molecules.

In the embodiment of the present invention, the channel length of the MOSFET (i.e., the gate length L in FIG. 1) is set equal to or longer than the gate width W. Since nucleic-acid-probe molecules 102 are immobilized along the channel (i.e., in the direction in which the source 103 and drain 104 are connected to each other), the modulation of electrical properties of the MOSFET can reliably be induced even though a target nucleic acid molecule 109 is hybridized with one of the nucleic-acid-probe molecules 102 in any position along the channel. In other words, the circuit performs an operation that is equivalent to the logical OR operation between the nucleic-acid-probe molecules 102. Moreover, a possibility of hybridization of target and probe molecules is increased by densely arranging the sensors 100 within the chip surface on which the drops of a sample to be analyzed contact. Even few target nucleic acid molecules in the sample can thus be detected quickly.

There now follows a more specific description of how long and how wide the gate 101 is set. If the target nucleic acid molecule 109 is hybridized with a nucleic acid probe molecule 102, the variation in the number of charges on the gate 101 causes charge of potential in the channel through the gate oxide film 105. The Debye length of the carriers in the particular region of the body 106, where a channel is formed,
(ε₀εᵣk_{B}T/e²n)^{1/2} (E1)
where ε₀ is the dielectric constant of a vacuum, εᵣ is the relative dielectric constant of the channel region, k_{B} is the Boltzmann constant, T is the absolute temperature of the channel region, e is elementary charge, and n is an equilibrium carrier density in the corresponding region. When univalent charges vary on the gate 101, it is expected that the potential in a circle whose radius corresponds to the Debye length, which is given by the above expression (E1), in the channel region will vary greatly.

Determining the gate width and the gate length of the gate 101 which are equal to the length (Debye length) given by the above expression (E1), it is expected that the electrical properties of the MOSFET are greatly modulated by a small number of target nucleic acid molecules. The gate width is of the order of the length obtained by the expression (E1) and so is the gate length. In other words, the gate width and the gate length are each set to the length of almost the same figure (at most ten times or one-tenth) as that of the length obtained by the expression (E1). More favorably, the gate width is set to be of the order of the length obtained by the expression (El) and the gate length is set to be greater than the gate width.

With the material having the same carrier density as that of the feasible Si-MOSFET, for instance, 10¹⁵˜10¹⁶ cm⁻³ of an impurity concentration, the length obtained by the expression (E1) is about 50 nm and accordingly. Therefore, the gate width is set to 50 nm in the embodiment of the present invention. It does not matter that the gate width is about 100 nm, but more favorably it is about 50 nm or less. On the other hand, the gate length is equal to or greater than the gate width and thus it is about 50 nm or more.

The diameter of each of the nucleic acid molecules 102 is about 2 nm. When the molecules 102 are densely immobilized to the gate 101 whose gate width is 50 nm, twenty-five nucleic-acid probes are arranged across the channel. If a target nucleic acid molecule 109 whose length corresponds to about twenty base pairs is hybridized with one of the nucleic-acid-probe molecules 102 having the same length, charges are varied in accordance with the twenty base pairs. It is expected that the variation in charges will cause the physical properties of the MOSFET (e.g., variation in threshold voltage of the MOSFET) to vary greatly.

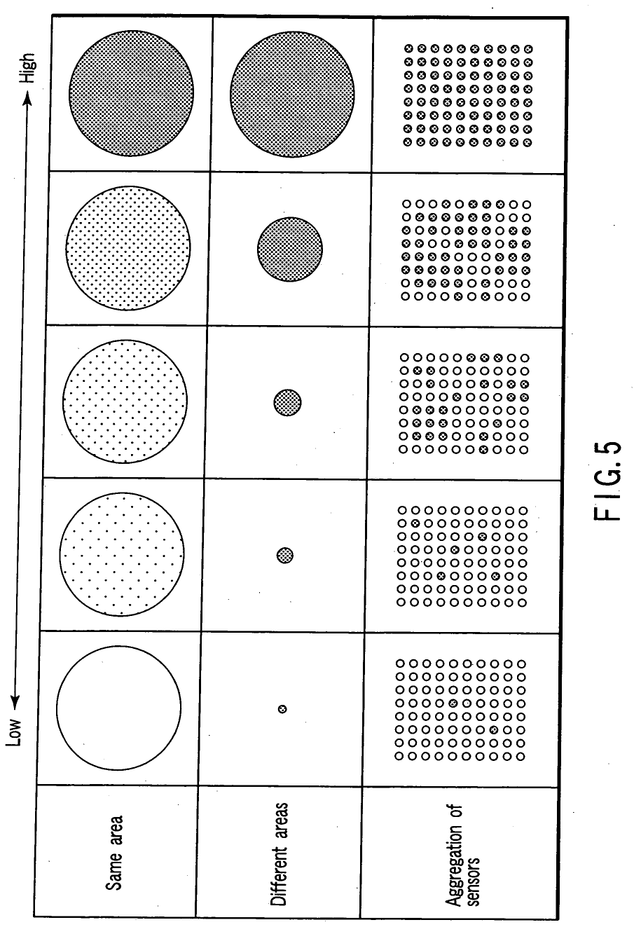

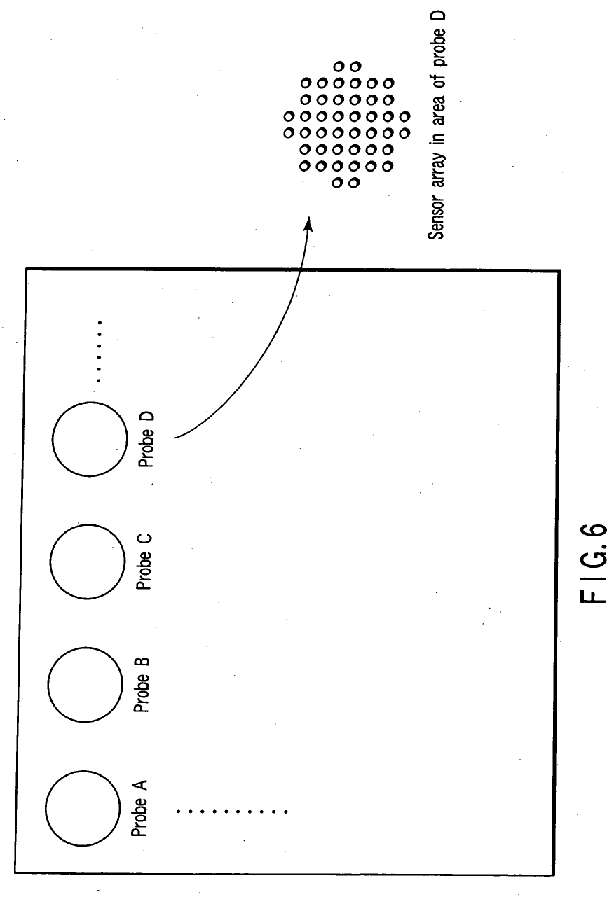

A plurality of nucleic-acid detecting sensors 100 are arranged on the chip. The precision with which a target nucleic acid molecule is detected varies with how the sensors 100 are arranged on the chip. Since the sensors 100 are densely arranged within the surface of the chip that the drops of a sample to be analyzed contact, the possibility that the target nucleic acid molecule will be hybridized with any one of a number of nucleic acid probe molecules. Even though there are few target nucleic acid molecules in the sample, they can be detected quickly. More favorably, the packing density of the sensors is so determined that the sensors can be arranged at intervals which are shorter than the diffusion distance of target nucleic acid molecules. Counting the number of sensors that have detected target nucleic acid molecules, the density of the target nucleic acid molecules can be estimated, as can be the number of target nucleic acid molecules. The arrangement of the sensors will be described in detail later with reference to FIGS. 5 and 6.

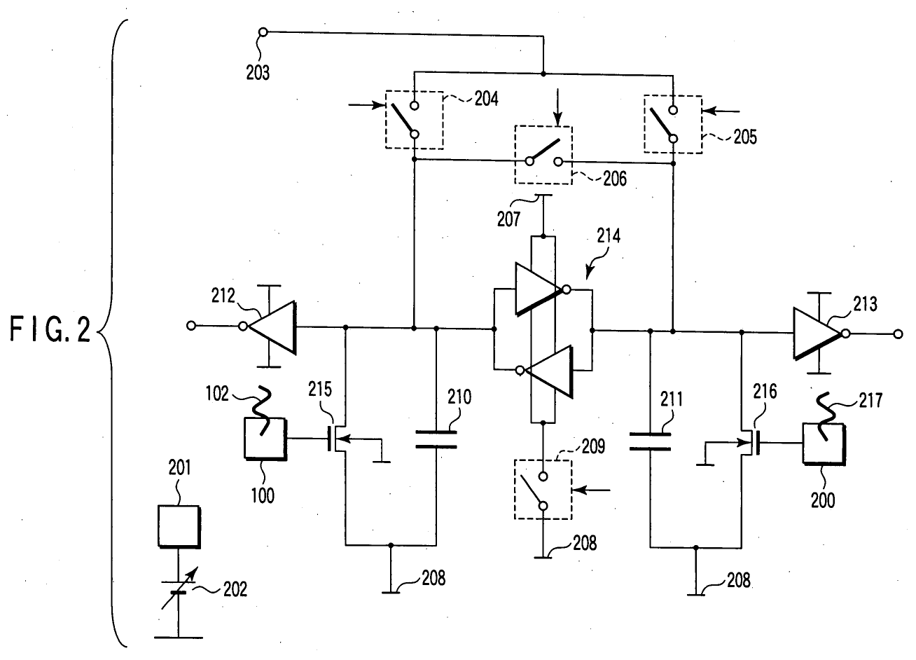

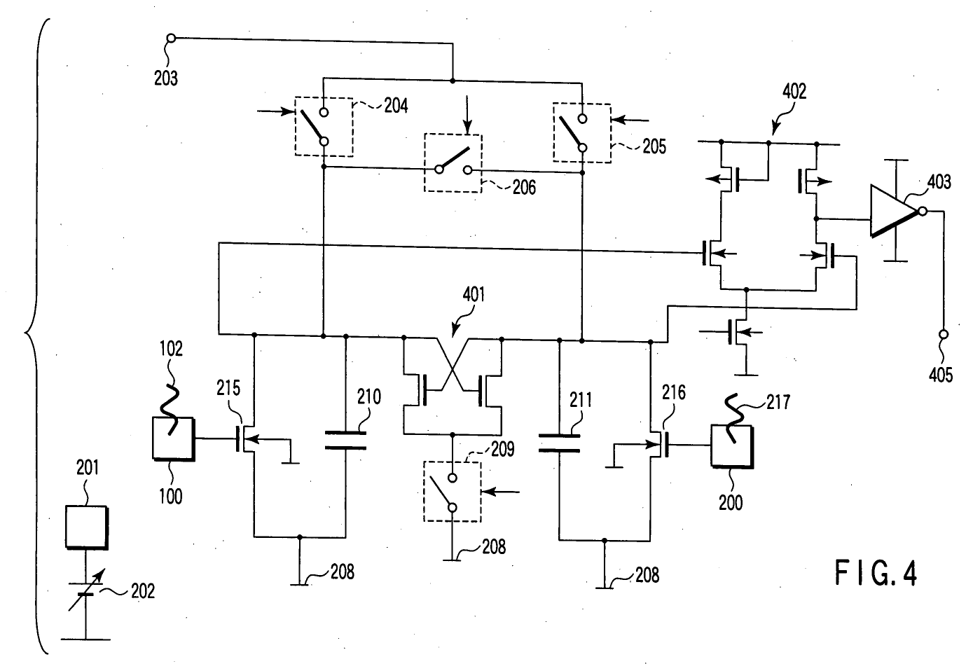

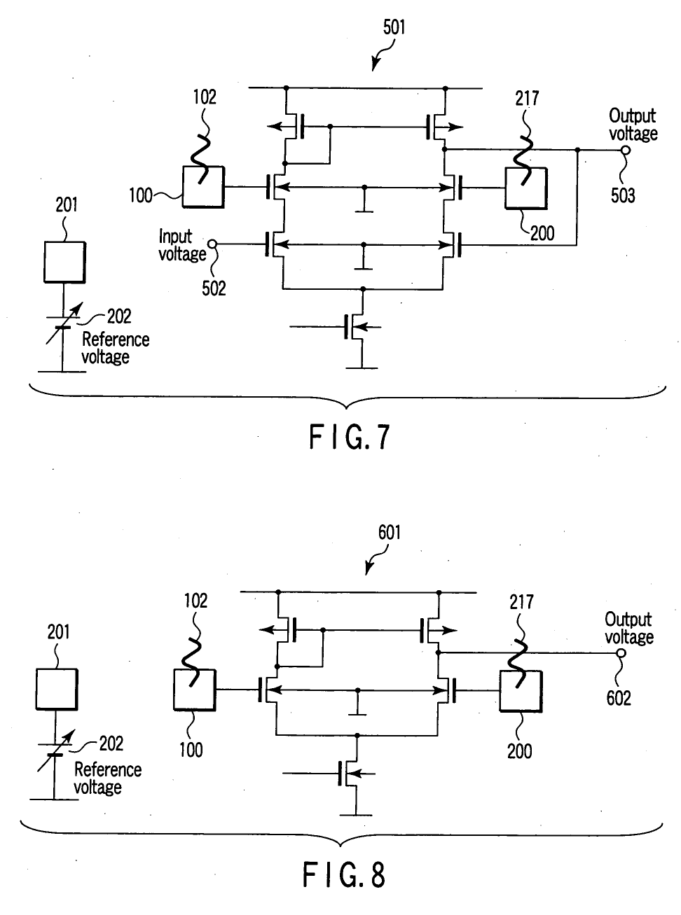

There will follows a description of a nucleic acid detecting circuit which detects the modulation of electrical properties of a MOSFET, which is induced by hybridization between a target nucleic acid molecule 109 and a nucleic acid probe molecule 102, using the above-described nucleic-acid detecting sensors 100. Since this modulation appears as a variation in threshold voltage, the nucleic acid detecting circuit detects this variation. In the embodiment of the present invention, two different nucleic acid detecting circuits for detecting the above physical phenomenon are provided as described below. One is a circuit (FIGS. 2 and 4) which directly converts a signal indicative of whether a target nucleic acid molecule 109 is detected or not into a digital signal and outputs the digital signal, and the other is a circuit (FIGS. 7, 8 and 9) which outputs a variation in threshold voltage as an analog voltage value. The feature of these two circuits is that the detection of a target nucleic acid molecule is determined by comparing the nucleic-acid detecting sensors 100 with zero-level detecting sensors on which nucleic acid probe molecules free of a base sequence complementary to the target nucleic acid molecule 109 are immobilized. With this feature, the target nucleic acid molecule 109 can be detected with higher precision.

An example of the nucleic acid detecting circuit which detects a target nucleic acid molecule using the nucleic acid detecting sensor 100 shown in FIG. 1 will be described with reference to FIG. 2. The nucleic acid detecting circuit shown in FIG. 2 employs a cross-coupled inverter.

Referring to FIG. 2, the nucleic acid detecting circuit includes a nucleic acid detecting sensor 100, a nucleic acid detecting sensor 200, a reference electrode 201, a reference voltage supply 202, a charging voltage supply input terminal 203, charging switches 204 and 205, a control pulse input terminal 206, a power supply voltage 207, a reference potential 208, a sense amplifier control switch 209, capacitors 210 and 211, output signal amplifiers 212 and 213 and a sense amplifier 214. The sensor 100 includes a MOSFET 215, and the sensor 200 includes a MOSFET 216 and a nucleic acid probe molecule 217.

The circuit shown in FIG. 2 includes a circuit which determines whether the threshold voltage of the MOSFET 215 included in the nucleic acid detecting sensor 100 on which the nucleic acid probe molecule 102 is immobilized has varied or not. This circuit is equivalent to a circuit used for reading data from a flash memory, and the MOSFET 215 corresponds to a MOSFET having a floating gate used in the flash memory. The reference electrode 201 of the circuit controls the surface potential of the MOSFET 215. The nucleic acid probe molecule 102 capable of being hybridized with a target nucleic acid molecule 109 is immobilized to the nucleic acid detecting sensor 100, while a nucleic acid probe molecule 217 incapable of being hybridized with the target nucleic acid molecule 109 is immobilized to the nucleic acid detecting sensor 200 that is paired with the sensor 100. The sensor 200 is a zero-level detecting sensor. The zero-level detecting sensor 200 is the same as the nucleic acid detecting sensor 100 except that the nucleic acid probe molecule 217 is immobilized in place of the nucleic acid probe molecule 102.

In the circuit shown in FIG. 2, the sense amplifier 214 compares the discharge time of the capacitor 210, which depends on the saturation current that varies with the threshold voltage of the MOSFET, which varies according to whether a target nucleic acid molecule is hybridized with the nucleic acid detecting sensor 100, with that of the capacitor 210 which depends on the threshold voltage of the zero-level detecting sensor 200. The sense amplifier 214 senses which of the sensors 100 and 200 lowers a voltage first and then outputs 0/1 to the higher/lower voltage node when the difference of voltage becomes sufficiently large. The output signals are output to external circuits through amplifiers 212 and 213.

In order to make the presence and absence of a target nucleic acid molecule in the nucleic acid detecting sensor 100 correspond to digital values “0” and “1,” the ratio between the capacitors 210 and 211 is set in advance such that the discharge time of the zero-level detecting sensor 200 is just half the sum of the discharge time required when the target nucleic acid molecule 109 is bounded with the nucleic acid detecting sensor 100 and the discharge time required when it is not hybridized therewith. Since the discharge time depends on the potential of the reference electrode, the voltage value of the reference voltage supply 202 has to be set in advance. Summarizing the above, the following parameters have to be determined in advance in order to operate the circuit shown in FIG. 2.

(1) The capacitance ratio between the capacitors 210 and 211, and

(2) The voltage value of the reference voltage supply 202 that determines the potential of the reference electrode 201 with respect to on the reference potential 208.

The parameter (1) will be described in detail. Assuming that the time constant of discharge time of capacitor 210 required when hybridization is detected is τ₁′, the time constant of discharge time of capacitor 210 required when hybridization is not detected is τ₁, and the time constant of discharge time of capacitor 211 is τ₂, the following expression should be established.
τ₁′<τ₂<τ₁ (E2)
The expression (E2) is based on the assumption that the MOSFET 215 of the nucleic acid detecting sensor 100 is of an n type and its threshold voltage is lowered due to the effect of a positively-charged intercalating agent by hybridization. When no intercalating agent is used, the n-type MOSFET increases in threshold voltage and thus the inequality signs of the expression (E2) are inverted. It is more favorable that τ₂ be set to an intermediate value between τ₁ and τ₁′ as given by the following expression:
τ₂=(τ₁+τ₁′)/2 (E3)

The above expression (E2) and equation (E3) are converted to the capacitance ratio between the capacitors. Assuming here that the MOSFETs of the nucleic acid detecting sensor 100 and zero-level detecting sensor 200 operate in a saturated region, the current that flows through the sensor 100 is represented by the following equation (E4):
i=μCW(V_{GS}31 Vₜₕ)²/L (E4)
where C is the capacity of an oxide film of the MOSFET, μ is surface channel mobility, W is a gate width, L is a gate length, V_{GS} is a gate-to-source voltage or a voltage between a reference electrode 201 and the source 103, and Vₜₕ is a threshold voltage of the MOSFET, which varies according to whether hybridization is detected or not. Assuming that a threshold voltage obtained when hybridization is detected is Vₜₕ′, a threshold voltage obtained when hybridization is not detected is Vₜₕ, and the currents corresponding to these voltage values are i′ and i, τ₁′, τ₁ and τ₂ are approximated as follows:
τ₁′=C₁₀Vₚᵣₑ/′
τ₁=C₁₀Vₚᵣₑ/I
τ₂=C₁₁Vₚᵣₑ/i (E5)
where C₁₀ and C₁₁ represent the capacity of the capacitor 210 and that of the capacitor 211, respectively, and Vₚᵣₑ represents a voltage value input from the charging voltage supply input terminal 203. Substituting the equations (E4) and (E5) into the expression (E2), the conditions that C₁₀ and C₁₁ are to satisfy are determined as follows.
1<C₁₀/C₁₁<(V_{GS}−Vₜₕ′)2/(V_{GS}−Vₜₕ)² (E6)
Using the equations (E4) and (E5), the condition given by the equation (E3) is more favorably determined as follows.
C₁₀/(2C₁₁−C₁₀)=(V_{GS}−Vₜₕ′)²/(V_{GS}−Vₜₕ)² (E7)

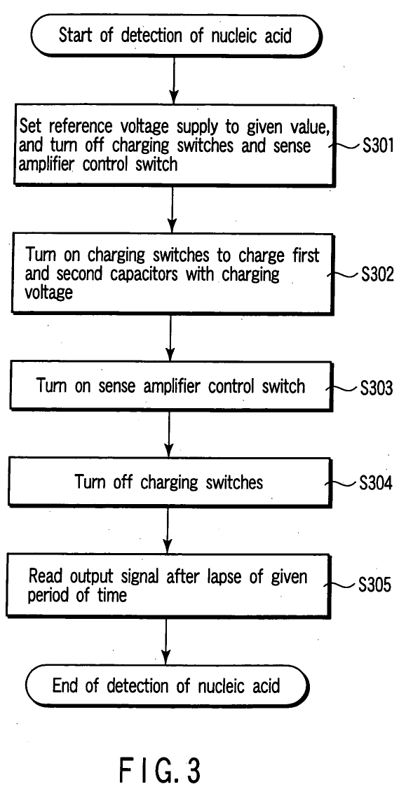

A procedure for detecting a nucleic acid using the circuit shown in FIG. 2 will be described with reference to FIG. 3.

First, a controller (not shown) turns off the charging switches 204 and 205 that determine whether to charge the capacitors 210 and 211 (step S301). The controller also turns off the sense amplifier control switch 209 that controls the sense amplifier 214 (step S301). Furthermore, the controller controls the reference voltage supply 202 as initialization such that the voltage between the reference electrode 201 and the source 103 of the nucleic acid detecting sensor 100 satisfies the above expression (E6) or equation (E7) (step S301).

The charging switches 204 and 205 turn on to apply a charging voltage to each of the capacitors 210 and 211 via the charging voltage supply input terminal 203 (step S302). Since the voltages applied to the capacitors 210 and 211 have the same value, the charges of the same quantity are stored in the capacitors 210 and 211. After that, the sense amplifier control switch 209 turns on to operate the sense amplifier 214 (step S303).

The charging switches 204 and 205 are turned off (step S304) to determine whether a nucleic acid is detected or not in accordance with a digital value “0” or “1” sensed by the sense amplifier 214 after a lapse of a given period of time. A normal operation can be performed even if the steps S304 and S305 can be changed to each other.

An example of a modification to the circuit shown in FIG. 2 will be described with reference to FIG. 4. The same components as those of FIG. 2 are denoted by the same reference numerals and their descriptions are omitted.

The modification shown in FIG. 4 includes a sense amplifier 401 that is obtained by forming the sense amplifier 214 of an nMOS only. The operating principle of the modification is basically the same as that of the circuit shown in FIG. 2, but a differential amplifier 402 has to be added thereto. In the modification, the nodes to which the capacitors 210 and 211 are connected converge on a potential between voltage value Vₚᵣₑ input from the charging voltage supply input terminal 203 and reference potential 208. A difference between the nodes is amplified by the differential amplifier 402 and then amplified by an output amplifier 403. Finally, a detectable digital value “0” or “1” can be output from an output signal terminal 405.

Since nucleic-acid detecting sensors 100 are densely arranged on a chip substrate, the density of a nucleic acid molecule can be analyzed. If the surface density of the nucleic-acid detecting sensors 100 is (Dt)⁻¹ or more where t is detection time and D is the diffusion constant (1.6×10^{31 6} cm²/s), the nucleic acid molecule can be detected within detection time t. In other words, the surface density of the nucleic-acid detecting sensors 100 is higher than the density at which at least one of the nucleic-acid detecting sensors 100 is included in a circle whose radius corresponds to the diffusion distance of the nucleic acid molecule. At this surface density, an array of the nucleic-acid detecting sensors 100 is so formed that the sensors 100 can be arranged within a region into which the drops of a sample are introduced.

If an array of the nucleic-acid detecting sensors 100 is formed on a chip substrate at the surface density of 10⁻⁶/cm², the distance between adjacent sensors is about 10 μm. In this case, index 1 that represents the length of a nucleic acid molecule is obtained by the following equation (E8).
1=(Dt)^{1/2} (E8)
In the equation (E8), t is several seconds and thus the nucleic acid molecule can be detected at least in several minutes. In other words, it is expected that the nucleic acid molecule will be hybridized with any one of the sensors. If the surface density of the sensors is increased, the nucleic acid molecule can be detected at high speed.

A high-speed quantitative analysis can be conducted using a chip on which the nucleic-acid detecting sensors 100 are densely arranged. The method described so far is disclosed in, for example, Jpn. Pat. Appln. KOKAI Publication No. 2004-309462 described above. The method is proposed as follows. A signal is generated by hybridizing only some of a number of nucleic acid probe molecules, which are included in a large sensor as shown in the upper row in FIG. 5, with target nucleic acid molecules. In order to prevent this signal from being hidden under a background signal, a small sensor is used as shown in the middle row in FIG. 5, and nucleic acid probe molecules are concentrated thereon and hybridized with target nucleic acid molecules. Though the method allows the target nucleic acid molecules to be detected sensitively, it has the drawback that response time has to be lengthened to enhance the sensitivity of the detection.

In the embodiment of the present invention, a high-speed quantitative analysis can be expected since target nucleic acid molecules have only to be hybridized with any of the sensors as shown in the lower row in FIG. 5, unlike in the above method. In the quantitative analysis, the number of sensors that are determined to detect target nucleic acid molecules in accordance with digital values is counted. The higher the density of the target nucleic acid molecules, the larger the number of sensors that have detected the target nucleic acid molecules. Based on the ratio of the number of sensors that have detected the target nucleic acid molecules to the total number of sensors, the density of the target nucleic acid molecules included in a sample is estimated.

When a quantitative analysis is conducted for different nucleic acids, a plurality of arrays of sensors to which nucleic acid probes having a base sequence are immobilized are arranged on the surface of a chip substrate, as shown in FIG. 6. A sample has to be introduced into the arrays.

Needless to say, nucleic-acid-probe molecules 102 of the same type need not be arranged together in one place. They can be arranged regularly or randomly since a quantitative analysis can be conducted if the surface density of the sensors is fixed.

(Modification to Embodiment of the Present Invention)

Another nucleic acid detecting circuit using a differential amplifier will be described with reference to FIGS. 7 and 8.

The circuit shown in FIGS. 7 and 8 includes a circuit which determines whether the threshold voltage of a MOSFET included in the nucleic acid detecting sensor 100 to which a nucleic acid probe molecule 102 is immobilized has varied or not using a differential amplifier. In the nucleic acid detecting sensor 100 according to the foregoing embodiment of the present invention, too, the detection of a nucleic acid can be determined using a circuit having a differential amplifier. In other words, the MOSFET of the nucleic acid detecting sensor 100 and that of the zero-level detecting sensor 200 are provided in the differential amplifier.

As shown in FIG. 8, an output voltage generated by setting the potential of the reference electrode 201 to a given value can be measured or, as shown in FIG. 7, a nucleic acid can be detected as a variation of the offset voltage of a voltage follower circuit formed by inserting transistors which are more than those of FIG. 8 by two.

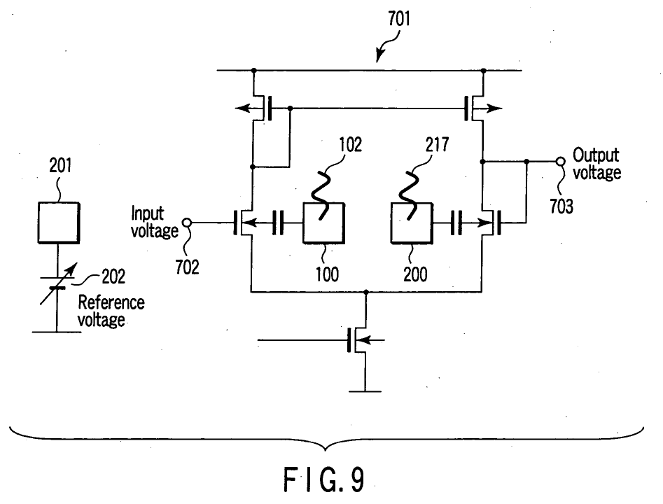

Even though a circuit having a double gate MOS structure capable of controlling the potential of a back gate is used as shown in FIG. 9, it can determine whether the threshold voltage of a MOSFET included in the nucleic acid detecting sensor 100 to which a nucleic acid probe molecule 102 is immobilized has varied or not. In other words, like in the circuit shown in FIG. 7, a variation of the offset voltage of a voltage follower circuit has only to be measured by controlling the potential of the nucleic acid detecting sensor 100 to which the nucleic acid probe molecule 102 is immobilized and that of the zero-level detecting sensor 200 through the reference electrode 201.

According to the foregoing embodiment of the present invention, the gate width of an FET of a nucleic acid detecting sensor is set to not larger than the Debye length of electrons in a channel region and the gate length thereof is set to not smaller than the Debye length, thereby increasing the sensitivity of detection drastically. Further, one nucleic acid molecule can be detected at very high speed. Since a plurality of nucleic acid detecting sensors are densely arranged on a detecting chip, a quantitative analysis can simultaneously be conducted within a very wide density range. High-precision detection can be carried out in a short time without amplification of nucleic acid such as polymerase chain reaction (PCR) or any indicators of a target nucleic acid molecule.

According to the nucleic acid detecting sensor, the nucleic acid detecting chip, and the nucleic acid detecting circuit, the sensitivity of detection can be improved drastically.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A nucleic acid detecting sensor comprising:
a field-effect transistor;
a detector which detects target nucleic acid molecules having sequences from a sample based on a degree of a variation in threshold voltage of the field-effect transistor; and
at least one nucleic acid probe molecule which is hybridized with a corresponding one of the target nucleic acid molecules, and is immobilized on a gate of the field-effect transistor,
wherein a gate width of the field-effect transistor is of an order of a length obtained by an expression given below:
(ε₀εᵣk_{B}T/e²n)^{1/2}
where ε₀ is a dielectric constant of a vacuum, εᵣ is a relative dielectric constant of a channel region, k_{B} is a Boltzmann constant, T is an absolute temperature of the channel region, e is elementary charge, and n is an equilibrium carrier density in the channel region in the field-effect transistor where a channel is formed.

2. The sensor according to claim 1, wherein a gate length of the field-effect transistor is of the same order as that of the gate width of the field-effect transistor and is greater than the gate width thereof.

3. A nucleic acid detecting sensor comprising:
a field-effect transistor;
a detector which detects target nucleic acid molecules having sequences from a sample based on a degree of a variation in threshold voltage of the field-effect transistor; and
at least one nucleic acid probe molecule which is hybridized with a corresponding one of the target nucleic acid molecules, and is immobilized on a gate of the field-effect transistor,
wherein a gate length of the field-effect transistor is of an order of a length obtained by an expression given below:
(ε₀εᵣk_{B}T/e²n)^{1/2}
where ε₀ is a dielectric constant of a vacuum, εᵣ is a relative dielectric constant of a channel region, k_{B} is a Boltzmann constant, T is an absolute temperature of the channel region, e is elementary charge, and n is an equilibrium carrier density in the channel region in the field-effect transistor where a channel is formed.

4. A nucleic acid detecting chip including a plurality of nucleic acid detecting sensors according to claim 1,
wherein number of nucleic acid detecting sensors per unit area on the nucleic acid detecting chip is of an order that is equal to or greater than that of a value obtained by an expression given below:
1/Dt
where t is specified detection time and D is a diffusion constant of a nucleic acid molecule.

5. The nucleic acid detecting chip according to claim 4, wherein a density of target nucleic acid molecules included in the sample is estimated based on a ratio of the number of nucleic acid detecting sensors, which have detected the target nucleic acid molecules, to total number of nucleic acid detecting sensors.

6. A nucleic acid detecting chip including a plurality of nucleic acid detecting sensors according to claim 2,
wherein number of nucleic acid detecting sensors per unit area on the nucleic acid detecting chip is of an order that is equal to or greater than that of a value obtained by an expression given below:
1/Dt
where t is specified detection time and D is a diffusion constant of a nucleic acid molecule.

7. The nucleic acid detecting chip according to claim 6, wherein a density of target nucleic acid molecules included in the sample is estimated based on a ratio of the number of nucleic acid detecting sensors, which have detected the target nucleic acid molecules, to total number of nucleic acid detecting sensors.

8. A nucleic acid detecting chip including a plurality of nucleic acid detecting sensors according to claim 3,
wherein number of nucleic acid detecting sensors per unit area on the nucleic acid detecting chip is of an order that is equal to or greater than that of a value obtained by an expression given below:
1/Dt
where t is specified detection time and D is a diffusion constant of a nucleic acid molecule.

9. The nucleic acid detecting chip according to claim 8, wherein a density of target nucleic acid molecules included in the sample is estimated based on a ratio of the number of nucleic acid detecting sensors, which have detected the target nucleic acid molecules, to total number of nucleic acid detecting sensors.

10. A nucleic acid detecting circuit comprising:
a nucleic acid detecting sensor according to claim 1;
a zero-level detecting sensor having a gate on which a nucleic acid probe molecule is immobilized, the nucleic acid probe molecule differing from a nucleic acid probe molecule immobilized to the nucleic acid detecting sensor and having a sequence that fails to be complementary to nucleic acid molecules included in the sample;
two capacitive elements connected to a drain terminal of the nucleic acid detecting sensor and a drain terminal of the zero-level detecting sensor, respectively;
a sense amplifier which amplifies a difference in discharge rate between the field-effect transistor of the nucleic acid detecting sensor and that of the zero-level detecting sensor while those field-effect transistors discharge the capacitive elements charged with a present voltage; and
a determination unit configured to determine whether a target nucleic acid molecule is detected based on the difference in discharge efficiency.

11. A nucleic acid detecting circuit comprising:
a nucleic acid detecting sensor according to claim 2;
a zero-level detecting sensor having a gate on which a nucleic acid probe molecule is immobilized, the nucleic acid probe molecule differing from a nucleic acid probe molecule immobilized to the nucleic acid detecting sensor and having a sequence that fails to be complementary to nucleic acid molecules included in the sample;
two capacitive elements connected to a drain terminal of the nucleic acid detecting sensor and a drain terminal of the zero-level detecting sensor, respectively;
a sense amplifier which amplifies a difference in discharge rate between the field-effect transistor of the nucleic acid detecting sensor and that of the zero-level detecting sensor while those field-effect transistors discharge the capacitive elements charged with a present voltage; and
a determination unit configured to determine whether a target nucleic acid molecule is detected based on the difference in discharge efficiency.

12. A nucleic acid detecting circuit comprising:
a nucleic acid detecting sensor according to claim 3;
a zero-level detecting sensor having a gate on which a nucleic acid probe molecule is immobilized, the nucleic acid probe molecule differing from a nucleic acid probe molecule immobilized to the nucleic acid detecting sensor and having a sequence that fails to be complementary to nucleic acid molecules included in the sample;
two capacitive elements connected to a drain terminal of the nucleic acid detecting sensor and a drain terminal of the zero-level detecting sensor, respectively;
a sense amplifier which amplifies a difference in discharge rate between the field-effect transistor of the nucleic acid detecting sensor and that of the zero-level detecting sensor while those field-effect transistors discharge the capacitive elements charged with a present voltage; and
a determination unit configured to determine whether a target nucleic acid molecule is detected based on the difference in discharge efficiency.

13. A nucleic acid detecting circuit comprising:
a nucleic acid detecting sensor according to claim 1;
a zero-level detecting sensor having a gate on which a nucleic acid probe molecule is immobilized, the nucleic acid probe molecule differing from a nucleic acid probe molecule immobilized to the nucleic acid detecting sensor and having a sequence that fails to be complementary to nucleic acid molecules included in the sample;
a differential pair using the field-effect transistor of each of the nucleic acid detecting sensor and the zero-level detecting sensor as an input transistor; and
a determination unit configured to determine whether a target nucleic acid molecule is detected based on an intensity of an output voltage of the differential pair, which is generated by applying a common reference voltage to the differential pair.

14. A nucleic acid detecting circuit comprising:
a nucleic acid detecting sensor according to claim 2;
a zero-level detecting sensor having a gate on which a nucleic acid probe molecule is immobilized, the nucleic acid probe molecule differing from a nucleic acid probe molecule immobilized to the nucleic acid detecting sensor and having a sequence that fails to be complementary to nucleic acid molecules included in the sample;
a differential pair using the field-effect transistor of each of the nucleic acid detecting sensor and the zero-level detecting sensor as an input transistor; and
a determination unit configured to determine whether a target nucleic acid molecule is detected based on an intensity of an output voltage of the differential pair, which is generated by applying a common reference voltage to the differential pair.

15. A nucleic acid detecting circuit comprising:
a nucleic acid detecting sensor according to claim 3;
a zero-level detecting sensor having a gate on which a nucleic acid probe molecule is immobilized, the nucleic acid probe molecule differing from a nucleic acid probe molecule immobilized to the nucleic acid detecting sensor and having a sequence that fails to be complementary to nucleic acid molecules included in the sample;
a differential pair using the field-effect transistor of each of the nucleic acid detecting sensor and the zero-level detecting sensor as an input transistor; and
a determination unit configured to determine whether a target nucleic acid molecule is detected based on an intensity of an output voltage of the differential pair, which is generated by applying a common reference voltage to the differential pair.
